(19) United States
(12) Patent Application Publication
(10) Pub. No.: US 2006/0104961 A1
(43) Pub. Date: May 18, 2006

(54) Mechanisms of myoblast transfer in treating heart failure

(76) Inventor: Peter K. Law, Ontario (CA)

Correspondence Address: HELLER EHRMAN WHITE & MCAULIFFE LLP, 1717 RHODE ISLAND AVE, NW, WASHINGTON, DC, 20036-3001, US

(21) Appl. No.: 10/523,969

(22) PCT Filed: Aug. 6, 2003
(86) PCT No.: PCT/US03/24600

§ 371 Date: Oct. 7, 2005

Related U.S. Application Data
(60) Provisional application No. 60/402,050, filed on Aug. 9, 2002.

(51) Int. Cl. A61K 48/00; C12N 5/08

(52) U.S. Cl. 424/93.21; 435/366

(57) ABSTRACT

Bioengineering the regenerative heart provides a novel treatment for heart failure. On May 14, 2002, a 55-year-old man suffering ischemic myocardial infarction received 25 injections carrying 465 million cGMP-produced pure myoblasts into his myocardium after coronary artery bypass grafting. Three myogenesis mechanisms were elucidated with 17 human/porcine xenografts using cyclosporine as immunosuppressant. Some myoblasts developed to become cardiomyocytes. Others transferred their nuclei into host cardiomyocytes through natural cell fusion. As yet others formed skeletal myofibers with satellite cells. De novo production of contractile filaments augmented heart contractility. Human myoblasts transduced with VEGF₁₆₅ gene produced six times more capillaries in porcine myocardium than placebo. Xenograft rejection was not observed for up to 20 weeks despite cyclosporine discontinuation at 6 weeks.

This application claims priority to U.S. provisional application Ser. No. 60/402,050, filed Aug. 9, 2002, the entirety of which is hereby incorporated by reference.

### FIELD OF THE INVENTION

The invention relates generally to treatment of infarcted myocardium and more specifically to the use of cell therapy to repair myocardial infarction through concomitant angiogenesis and myogenesis.

### BACKGROUND OF THE INVENTION

Heart muscle degeneration is the leading cause of debilitation and death in humans. It cascades in losses of live cardiomyocytes, contractile filaments, and heart function. Cardiomyocytes do not regenerate significantly because the telomeric DNA repeats¹ in these terminally differentiated cells are minimal.

The degenerative heart transmits biochemical signals to recruit stem cells to repair the muscle damage. Being pluripotent, embryonic or adult stem cells exhibit uncontrolled differentiation into various lineages to produce bone, cartilage, fat, connective tissue, skeletal and heart muscles (FIG. 1). The damaged myocardium needs additional live myogenic cells to deposit contractile filaments to regain heart function, preferably before fibroblast infiltration which leads to scar formation. However, until scientists can accurately define the specific transcriptional factors and pathway to guide stem cell differentiation into cardiomyocytes, the use of stem cell injection into the human heart would have risk-benefit ratio higher than the use of myoblasts.

As young cardiomyocytes and myoblasts become committed and differentiate from stem cells, they are similar in that they are mononucleated cells without contractile filaments. In the presence of neurotrophic factors, myoblasts fuse to become myotubes that develop into myofibers. Under the influence of heart hormones, the young cardiomyocytes fuse to become mature cardiomyocytes. Cardiomyocytes and myofibers are myogenic cells that produce contractile proteins to provide contractility.

Like cardiomyocytes, myoblasts are differentiated cells that are destined to become muscles. Unlike cardiomyocytes however, myoblasts have long telomeric DNA subunits and are capable of extensive mitosis. The ability to undergo mitosis and to fuse are conserved in mononucleated satellite cells that are essentially myoblast reserves in adult muscles. Satellite cells are differentiated cells. They are not stem cells. Myoblasts survive and proliferate in intercellular fluid. Their survival does not depend on vascularization or nerve innervation.

Because of these desirable properties of myoblasts, attempts have been made to repair muscle tissue with them. As shown by the work of Law et al. an important feature in this regard is that the myoblast preparations used for cell transplantation should be relatively free of fibroblasts, an important point that seems to have been overlooked by many workers in this field. See for example, Law et al. Gene Therapy and Molecular Biology Vol. 1, 345-363, particularly page 350 to 351, describing myogenic cell transplant therapy for correction and amelioration of skeletal muscle disorders. Another point is that once introduced, the myoblasts are subjected to scavenger hunt by macrophages, typically for up to three weeks. Thus, a high number of myoblasts must be transplanted for the transplanted cells to be effective.

The first human myoblast transfer into the porcine heart revealed that it was safe to administer one billion myoblasts at 100×10⁶/ml through the Myostar catheter (Biosense Webster, Inc.) using 20 injections at different locations inside the left ventricle.² It was determined that 0.3 ml to 0.5 ml would be the optimal volume per injection. This field of medicine has become very active. However, generally acceptable and successful results remain elusive. More needs to be done to obtain successful transfer of cells into damaged hearts in a manner that corrects the damage.

### SUMMARY OF THE INVENTION

Embodiments of the invention allow cell therapy of diseased heart tissue. In one embodiment a composition is provided that comprises isolated myoblasts that transgenically express VEGF. Another embodiment provides a composition wherein the number of myoblasts exceed the number of fibroblast cells by 100 to 1. Yet another embodiment provides a composition that comprises isolated myoblasts cotransfected with a gene that encodes an epithelial cell stimulator or angiogenesis stimulator and a second marker gene. Yet another embodiment provides a composition useful for alleviating congestive heart failure, comprising at least 1 billion myogenic cells that transgenically express at least one angiogenesis factor. In yet another embodiment the cells transgenically express VEGF 165. In yet another embodiment the cells further express VPF.

Another embodiment provides a method for treating congestive heart failure in an individual, comprising taking a biopsy of skeletal muscle from the individual to form a culture; transforming cells of the culture with at least one foreign gene that encodes an angiogenesis factor; forming a culture of cells suitably pure enough for repairing the heart of the individual; and introducing cells of the culture into a diseased heart of the individual.

### DESCRIPTION OF THE FIGURES

FIG. 1. Advantages of using myoblasts over stem cells in treating heart failure. MTT=myoblast transfer therapy

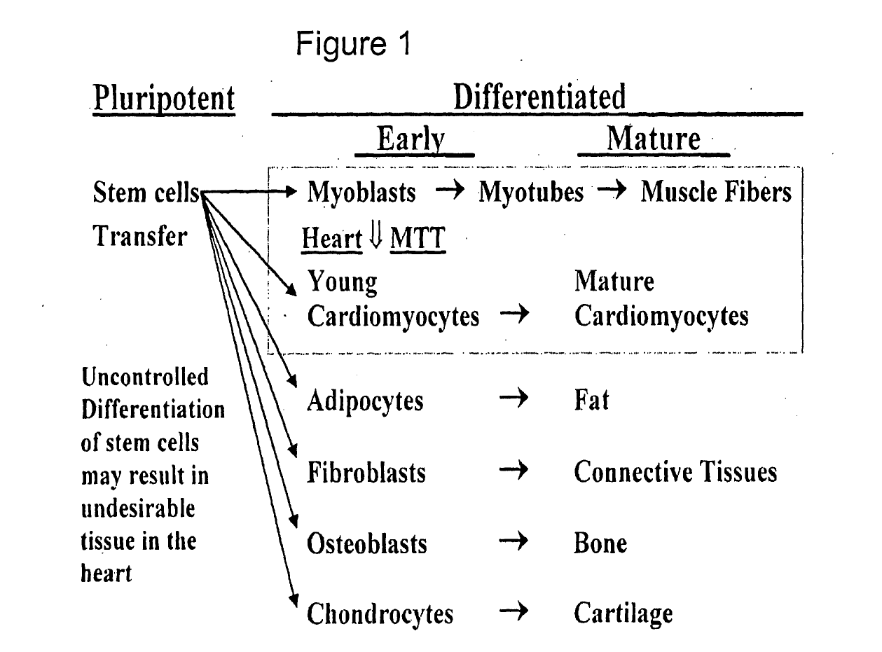

FIG. 2. Human desmin immunostain for myoblast purity. (A) Positive control of leiomyosarcoma, desmin staining brown. (B) Negative control. (C) Pure human myoblasts immunostained with desmin. (D) Pure human myoblasts in culture.

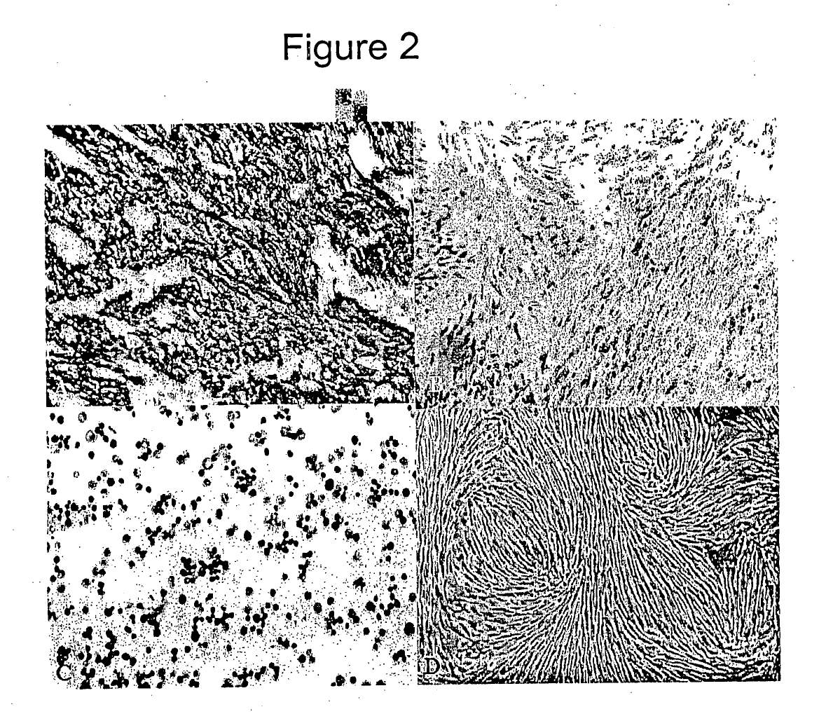

FIG. 3. (A) Brownish immunostain of human myosin in porcine myocardium 12 weeks after human myoblast injection. (B) Cardiomyocytes with Lac-Z positive nuclei and human myosin stain, indicative of donor or myoblastic in origin. (C) Negative immunostain (grey) of human myosin in porcine myocardium sham-injected without myoblasts.

FIG. 4. (A) Heterokaryons derived from fusion of porcine cardiomyocytes and human myoblasts showing Lac-Z positive human myoblast nuclei (bluish green) and porcine cardiomyocyte nuclei (purple) in the heterokaryotic syncytium. (B) These heterokaryons expressed human myosin heavy chain.

FIG. 5. Electron microscopy of the myoblast-injected porcine myocardium showing (A) myotubes with central nuclei and myfibril (ML) deposits, and (B) skeletal myofiber with satellite cell (SC) and nucleus (N). The satellite cell was located between the basement membrane (black arrow) and the plasma membrane (white arrow). Sarcomeres showed proper alignment of newly formed contractile filaments.

FIG. 6. (A) Control myocardium immunostained for vWF VIII and counterstained with Eosin to show capillaries. (B) VEGF₁₆₅ transduced myoblasts produced increased vascular density. (C) As in B but without Eosin counterstain.

### DETAILED DESCRIPTION OF THE INVENTION

Myogenic cells according to embodiments of the invention preferably are autologous and obtained by tissue biopsy, as described for example in U.S. Pat. Nos. 6,099,832, 5,833,978, 6,284,242 and 5,130,141. In preferred embodiments, a composition is prepared from a myogenic cell or precursor to myogenic cell having a minimum contamination by fibroblasts. The term “minimum contamination by fibroblasts means that on a total cell number basis, less than 5%, 2%, 1% 0.5% 0.2% or even less than 0.1% of the cells are fibroblast cells. This improved composition purity can be achieve a variety of ways. For example, in one way, fibroblast cells are preferentially inhibited or killed by the inclusion of one or more substances in the culture medium. In another way, a cell sorter is used that separate cells one at a time. In yet another way, a non-fibroblast specific promotor such as a muscle specific promoter is used to control expression of a gene that generates a product which, allows a cell that makes that product survive in cell culture. In this way, transformed myogenic cells preferentially survive and the percentage of fibroblasts diminishes. In yet another way, the myogenic cells are cultured in the presence of a macrophage cytokine such as that described by Giurisato et al. in Basic Appl. Myol. 8(5): 381-388 (1998), which stimulates proliferation of myogenic cells but not fibroblast cells. The cytokine may be produced by culturing macrophages in a serum free medium and then harvesting the medium to obtain a crude preparation of cytokine. In a preferred embodiment basic cell transfer therapy techniques that utilize very purified (low fibroblast contamination) cultures are made possible by adding crude or partially purified preparations of a 50-10 KDa cytokine secreted by macrophages to the culture and growing at least 2, 3, 5, 8 or 10 generations or more of the myogenic cells.

In each case some cell division may be used to increase cell number prior to use of the cells. Most desirably at least 1, 2, 5, 10, 25, 50 or even more than 100 billion cells are cultured and transferred. Where the cells are not autologous, it is generally preferred to utilize an agent to minimize or eliminate xenograft rejection such as cyclosporine, in the animal or patient that receives the cells. Further agents may be added to the reintroduced cells such as viscosity adjusting materials, adhesive agents and the like, to assist placement and positioning of the cells within myocardium during or after cell transfer.

Myogenic cells, or precursors to myogenic cells are activated or transformed to express one or more genes that stimulate endothelial cell growth and/or development of blood vessels. According to an embodiment of the invention the cells before transfer into a diseased heart are transformed with one or more genes under the control of a suitable promotor that expresses an endothelial cell growth and/or angiogenesis protein. For example, both acidic and basic fibroblast growth factor molecules are mitogens for endothelial cells and other cell types and desirably are stably incorporated into cells that are or become myogenic cells. Angiotropin and angiogenin can induce angiogenesis, as described by Folknan, J., Cancer Medicine, Lea and Febiger Press, pp. 153-170 (1993). A highly selective mitogen for vascular endothelial cells is vascular endothelial growth factor or VEGF (Ferrara, N. et al., Endocr. Rev. 13:19-32 (1992)), which is also known as vascular permeability factor (VPF). In most preferred embodiments VEGF is transgenically expressed. However, in some embodiments the desired gene is turned on by homologous recombination. Most preferably VEGF is transgenically expressed. In an embodiment at least two different genes are transgenically expressed such as VEGF with angiotropin or angiogenin. In another emobodiment more than two different genes are transgenically expressed. Multiple genes can be expressed within the same cell, or may be expressed by different cells within the same composition. In some circumstances expression of two different factors, such as two different angiogenesis factors synergistically results in greater establishment of the transplanted cells within a target diseased heart muscle.

Transformation of cells can be achieved by a variety of methods as will be appreciated by a skilled artisan. Generally a nucleic acid sequence encoding a desired polypeptide such as the VEGF165 gene is under the control of a suitable promoter. Suitable promoters which may be employed include, but are not limited to, adenoviral promoters, such as the adenoviral major late promoter; or heterologous promoters, such as the cytomegalovirus (CMV) promoter; the respiratory syncytial virus (RSV) promoter; inducible promoters, such as the MMT promoter, the metallothionein promoter; heat shock promoters; the albumin promoter; the ApoAl promoter; human globin promoters; viral thymidine kinase promoters, such as the Herpes Simplex thymidine kinase promoter; retroviral LTRs (including the modified retroviral LTRs hereinabove described); the .beta.-actin promoter; and human growth hormone promoters. The promoter also may be the native promoter which controls the gene encoding the polypeptide.

In one embodiment a retroviral plasmid vector is employed to transduce packaging cell lines to form producer cell lines. Examples of packaging cells which may be transfected include, but are not limited to, the PE501, PA317, .psi.-2, .psi.-AM, PA12, T19-14X, VT-19-17-H2, .psi.CRE, .psi.CRIP, GP+E-86, GP+envAml12, and DAN cell lines as described in Miller, Human Gene Therapy 1:5-14 (1990), which is incorporated herein by reference in its entirety. The vector may transduce the packaging cells through any means known in the art. Such means include, but are not limited to, electroporation, the use of liposomes, and CaPO.sub.4 precipitation. In one alternative, the retroviral plasmid vector may be encapsulated into a liposome, or coupled to a lipid, and then administered to a host. In this embodiment the producer cell line generates infectious retroviral vector particles that include the nucleic acid sequence(s) encoding the polypeptides. Such retroviral vector particles then may be employed, to transduce myogenic cells or precursors to myogenic cells. The transduced cells express the nucleic acid sequence(s) encoding the polypeptide.

In a preferred embodiment cells are transformed with VEGF. VEGF has four different forms of 121, 165, 189 and 206 amino acids due to alternative splicing as described, for example in U.S. Pat. No. 6,040,157. VEGF121 and VEGF165 are soluble and are capable of promoting angiogenesis, whereas VEGF189 and VEGF206 are bound to heparin containing proteoglycans in the cell surface. The temporal and spatial expression of VEGF has been correlated with physiological proliferation of blood vessels (Gajdusek, C. M., and Carbon, S. J., Cell Physiol 139:570-579 (1989); McNeil, P. L., et al., J Cell. Biol. 109:811-822 (1989)). Its high affinity binding sites are localized only on endothelial cells in tissue sections (Jakeman, L. B., et aL., Clin. Invest. 89:244-253 (1989)). In an embodiment of the invention at least two types of cells are transplanted into differing regions of the heart. One type of cell expresses VEGF (and optionally another angiogenesis factor) and preferentially is transplanted into region(s) where blood vessel growth is most desired. A second type of cell is transplanted into region(s) where blood vessel growth is less needed. A cardiac specialist can readily determine optimized locations for transplanting the two (or more) types of cells.

Vascular permeability factor (VPF) has also been found responsible for persistent microvascular hyperpermeability to plasma proteins even after the cessation of injury, which is a characteristic feature of normal wound healing. This suggests that VPF is an important factor in myocardium wound healing. Brown, L. F. et al., J. Exp. Med. 176:1375-1379 (1992). VEGF expression is high in vascularized tissues, (e.g., lung, heart, placenta and solid tumors) and correlates with angiogenesis both temporally and spatially. VEGF also has been shown to induce angiogenesis in vivo. Since angiogenesis is essential for the repair of normal tissues, especially vascular tissues, VEGF has been proposed for use in promoting vascular tissue repair (e.g., in atherosclerosis).

U.S. Pat. No. 5,073,492, issued Dec. 17, 1991 to Chen et al., discloses a method for synergistically enhancing endothelial cell growth in an appropriate environment which comprises adding to the environment, VEGF, effectors and serum-derived factor. Also, vascular endothelial cell growth factor C sub-unit DNA has been prepared by polymerase chain reaction techniques. The DNA encodes a protein that may exist as either a heterodimer or homodimer. The protein is a mammalian vascular endothelial cell mitogen and, as such, is useful for the promotion of vascular development and repair, as disclosed in European Patent Application No. 92302750.2, published Sep. 30, 1992. In embodiments of the invention co-expression of VEGF165 with VPF and/or vascular endothelial cell growth factor C is used, in the same cells or in the same composition of cells, for desirable synergistic effects.

Prepared transgenic myogenic cell compositions further may comprise cell stimulatory agents and other materials to facilitate deposition and affixation of cells to the myocardium. Generally, the cells are introduced as a thick suspension by injection into the damaged myocardium.

The example describes a clinical trial that demonstrates with unequivocal evidence that cGMP-produced pure human myoblasts and proof of concept for Heart Cell Therapy.

Human myoblasts survived and integrated into the porcine ischemic myocardium, allowing concomitant cell therapy and gene therapy. Whereas the newly formed myofibers harbor satellite cells and impart regenerative capacity to the heart muscle, the genetic transformation of cardiomyocytes in vivo to become regenerative heterokaryons through myoblast genome transfer constitutes the ultimate heart repair. The regenerative heart³ also contains cardiomyocytes of myoblastic origin. In all three scenarios, new contractile filaments are deposited to improve heart contractility. This latter can be translated into the improvement in the quality of life of heart patients and in the prevention of heart attacks.

Accordingly pure (i.e. at least 95%, 97%, 98%, 99%, 99.5%, or even at least 99.7% pure) VEGF₁₆₅ myoblasts, when injected intramyocardially, are potential therapeutic transgene vehicles for concurrent angiogenesis and myogenesis to treat heart failure. Immunosuppression using cyclosporine for six weeks is effective for long term survival of xenografts or allografts.

Each document cited herein is specifically incorporated in its entirety by reference. The following example is presented by way of illustration and not by way of limitation.

### EXAMPLE

This example demonstrates cell therapy of myocardium damage using myogenic cells that transgenically express VEGF165. In this myogenesis example, cultured myoblasts derived from satellite cells of human rectus femoris biopsies were transduced with retroviral vector carrying Lac-Z reporter gene. Porcine heart model of chronic ischemia (n=9; control=3; myoblast implanted=6) was produced by clamping an ameroid ring around the left circumflex coronary artery. Four weeks later, each heart was exposed by left thoracotomy. Twenty injections (0.25 ml each) containing 300 million myoblasts, or 5 ml total volume of basal DMEM as control, were injected into the left ventricle intramyocardially. Left ventricular function was assessed using MIBI-Tc^{99m} SPECT scanning one week before injection to confirm myocardial infarction and at 6 weeks after injection.

Animals were maintained on cyclosporine at 5 mg/kg body weight from 5 days before, until 6 weeks after cell transplantation. The animals were euthanized at 6 weeks to 5 months post-operatively, and the heart was processed for histological, immunocytochemical and ultrastructural studies. Laser nuclear capture together with single nucleus RT-PCR was performed to delineate host and donor nuclei. In situ hybridization using fluorescent DNA probes specific for human Y-chromosome and chromosomes 1 & 10 for pig were used.

In the angiogenesis study, the human myoblasts were transduced with retroviral and adenoviral vectors carrying Lac-Z and human VEGF₁₆₅ genes, respectively. The cells were characterized for VEGF₁₆₅ transduction and expression efficiency by immunostaining, ELISA, immunoblotting and RT-PCR. A porcine heart model of infarction was created in eight female swines by left circumflex artery ligation. The animals were grouped as control (n=3) and myoblast-implanted (n=5). Angiography was performed to ensure complete occlusion of the blood vessel. Infarction was confirmed with MIBI-Tc^{99m} SPECT scanning. Four weeks later, 5 ml basal DMEM without or with 3×10⁸ human myoblasts carrying VEGF₁₆₅ and Lac-Z genes were injected into the left ventricle intramyocardially. The animals were maintained on cyclosporine (5 mg/kg body weight) for six weeks post-operatively. Hearts were then explanted and processed for immunocytochemical studies.

Human myoblasts of 99% purity determined by human desmin immunostaining were prepared. About 75% of the myonuclei were successfully transduced with retrovirus carrying Lac-Z gene. Trypan blue stain revealed >95% cell viability immediately before injection.

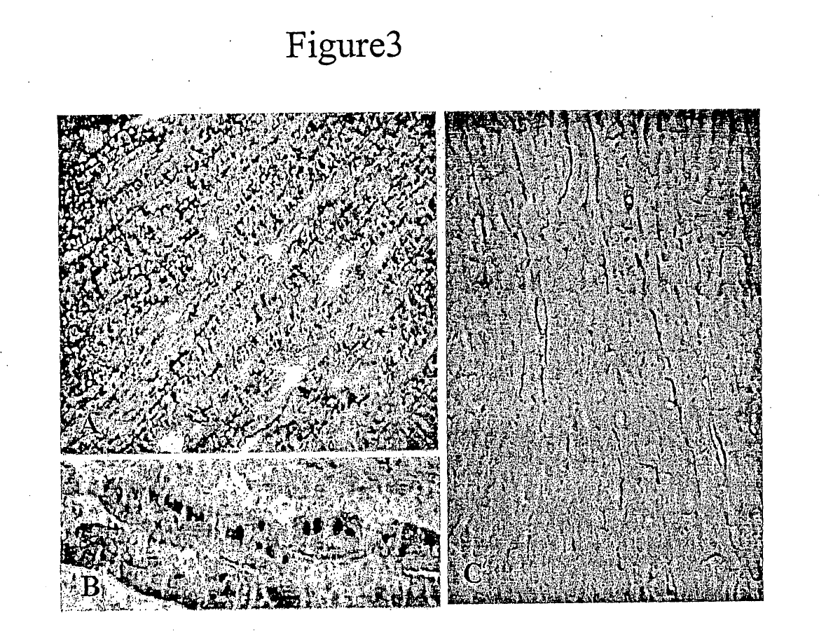

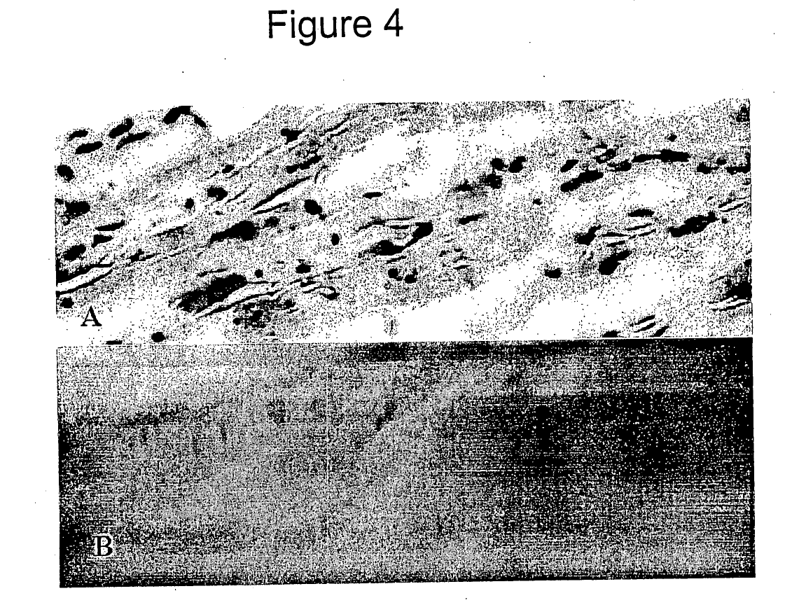

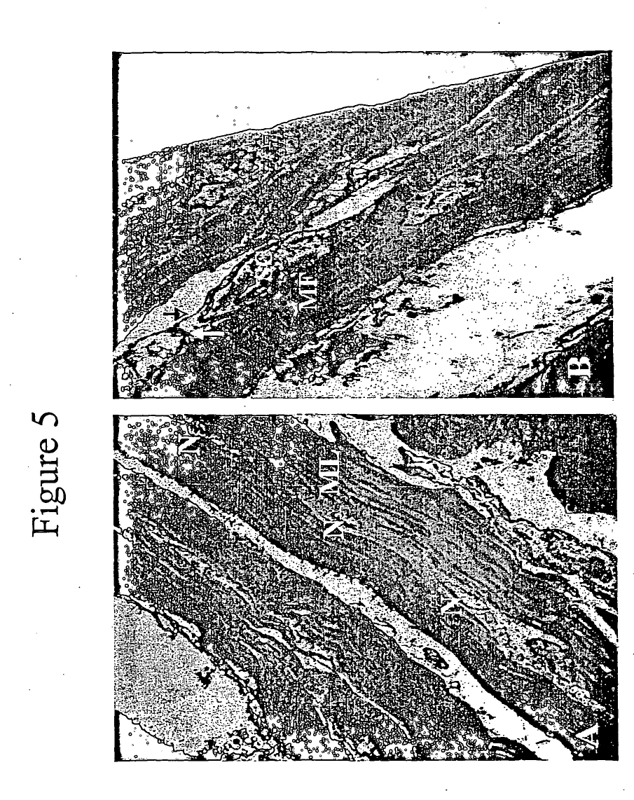

Histological examination of myoblast-injected myocardium showed cardiomyocytes containing Lac-Z positive nuclei (of donor origin) after 12 weeks (FIG. 3B). More than 80% of the Lac-Z positive cardiomyocytes immunostained positively for human myosin heavy chain (FIG. 3A). The control heart without myoblast injection did not show Lac-Z positive myonuclei nor human myosin (FIG. 3C). Triple stain of myoblast-injected myocardia demonstrated multinucleated heterokaryons containing human and porcine nuclei with expression of human myosin (FIG. 4). Electron microscopy demonstrated human myotubes and skeletal myofibers with satellite cells in the porcine myocardium (FIG. 5).

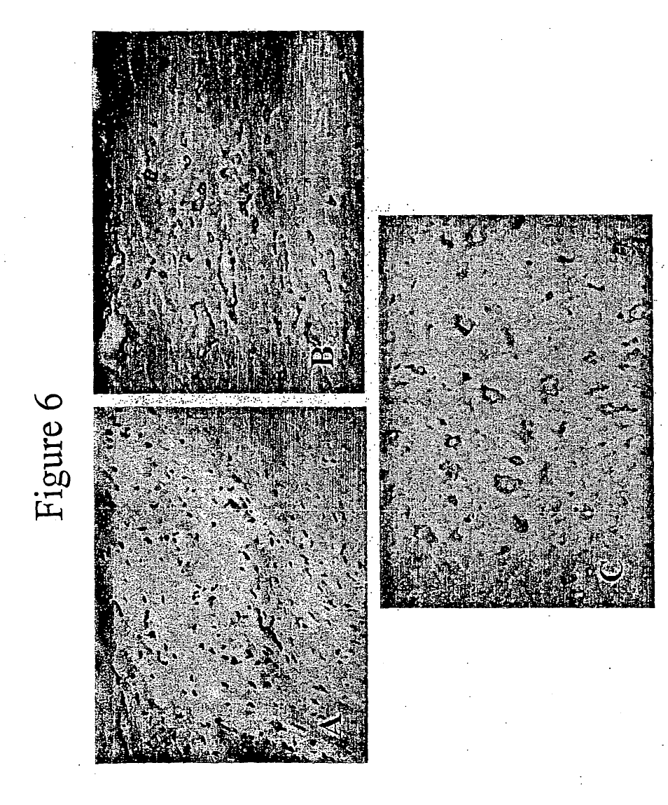

The transduction efficiency for Lac-Z and VEGF₁₆₅ was 75-80% and >95% respectively. The transduced myoblasts continued to secrete VEGF₁₆₅ for longer than 18 days, significantly higher (37±3 ng/ml) than non-transduced ones (200±30 pg/ml). Dye exclusion test reveals >95% cell viability at the time of injection. Histological examination showed extensive survival of the grafted myoblasts expressing Lac-Z gene in and around the infarct. The vascular density (mean±SEM) counted in an average of 12 low power fields (×200) in control animal hearts was (4.18+0.42) as compared to the VEGF₁₆₅ myoblast-transplanted group (28.31±1.84) (FIG. 6). The SPECT scans showed improved perfusion in the infarcted region. Discontinuation of cyclosporine after 6 weeks prompted no xenograft rejection for up to 20 weeks.

### FURTHER REFERENCES

1 F Ishikawa, M J Matunis, G Dreyfuss, and T R Cech, “Nuclear Proteins that Bind the Pre-mRNA 3′ Splice Site Sequence r(UUAG/G) and the Human Telomeric DNA Sequence d(TTAGGG)ₙ”, Molecular Cell Biology, (1993), 13, 4301-4310.
2 P K Law, et al., “World's First Human Myoblast Transfer into the Heart”, Frontiers in Physiology, (2000), p. A85.
3 P K Law, “The Regenerative Heart”, Pharma Tech 2002, 65-70.

Of course, changes and modifications to the embodiments presented herein are readily understood by the skilled artisan after reading this specification and furthermore, such changes and modifications may be practiced within the scope of the appended claims.

I claim:

1. A composition comprising isolated myoblasts that transgenically express an epithelial cell stimulator or angiogenesis stimulator.

2. The composition of claim 1, wherein the number of myoblasts exceed the number of fibroblast cells by 100 to 1.

3. The composition of claim 1, wherein the epithelial cell stimulator or angiogenesis stimulator is VEGF.

4. The composition of claim 3, wherein the VEGF is selected from the group consisting of VEGF2, VEGF121, VEGF165, or a biologically active fragment thereof.

5. The composition of claim 1, wherein the myoblasts further transgenically express at least a second epithelial cell stimulator or angiogenesis stimulator factor.

6. The composition of claim 5, wherein the second factor is selected from the group consisting of acidic fibroblast growth factor, basic fibroblast growth factor, angiotropin, angiogenin, and VPF.

7. The composition of claim 1, wherein the myoblasts are cotransfected with a gene that encodes an epithelial cell stimulator or angiogenesis stimulator and a second marker gene.

8. The composition of claim 1, comprising at least 1 billion myogenic cells that transgenically express at least one angiogenesis factor.

9. A composition as described in claim 8, wherein the at least one angiogenesis factor comprises VPF.

10. A method for treating congestive heart failure in an individual, comprising
1) taking a biopsy of skeletal muscle from the individual to form a culture;
2) transforming cells of the culture with at least one foreign gene that encodes an angiogenesis factor,
3) forming a culture of cells suitably pure enough for repairing the heart of the individual; and
4) introducing cells of the culture into a diseased heart of the individual.

11. The method of claim 10, wherein the culture of step 3 is at least 99% pure myoblasts.

12. The method of claim 10, wherein the at least one foreign gene comprises a VEGF polypeptide.

13. The method of claim 10, wherein the cells are introduced into the diseased heart by injections of at least 100 million cells each.

14. The method of claim 10, wherein at least one billion cells are introduced into the diseased heart.

15. A method for treating congestive heart failure in an individual, comprising
1) providing a culture of muscle cells;
2) transforming cells of the culture with at least one foreign gene that encodes an angiogenesis factor;
3) forming a culture of cells suitably pure enough for repairing the heart of the individual; and
4) introducing cells of the culture into a diseased heart of the individual.

16. The method of claim 15, wherein the culture of step 3 is at least 99% pure myoblasts.

16. The method of claim 15, wherein the at least one foreign gene comprises a VEGF polypeptide.

18. The method of claim 15, wherein the cells are introduced into the diseased heart by injections of at least 100 million cells each.

19. The method of claim 15, wherein at least one billion cells are introduced into the diseased heart.

20. The method of claim 15, wherein the individual is treated with cyclosporine prior to step 4.
